(19) United States
(12) Patent Application Publication
(10) Pub. No.: US 2004/0142887 A1
(43) Pub. Date: Jul. 22, 2004

(54) Antigen-polymer compositions

(76) Inventors: Chengji Cui, Ann Arbor (CN); Steven P. Schwendeman, Ann Arbor, MI (US); Vernon Stevens, Dublin, OH (US)

Correspondence Address: CALFEE HALTER & GRISWOLD, LLP, 800 SUPERIOR AVENUE, SUITE 1400, CLEVELAND, OH, 44114, US

(21) Appl. No.: 10/617,078

(22) Filed: Jul. 10, 2003

Related U.S. Application Data
(60) Provisional application No. 60/394,967, filed on Jul. 10, 2002.

(51) Int. Cl. A61K 48/00; A61K 39/00

(52) U.S. Cl. 514/44; 424/184.1; 424/185.1

(57) ABSTRACT

Methods for enhancing immunogenic response to an antigen, particularly a peptide antigen in a mammalian subject. The method comprises administering a biodegradable polymeric delivery system which comprises one or more antigens of interest and a biologically effective amount of one or more basic additives to the mammalian subject. In a highly preferred embodiment, the basic additive in MgCO₃, and the biodegradable polymeric delivery system is a PLGA microparticle. The present invention also relates to the immunogenic compositions used in the present method.

### CROSS REFERENCE TO RELATED APPLICATIONS

This application claims priority to U.S. Provisional Application Serial No. 60/394,967 filed Jul. 10, 2002, the entirety of which is incorporated herein by reference.

### STATEMENT REGARDING FEDERALLY FUNDED RESEARCH

This invention was made at least in part with government support under National Institutes of Health Grant HL 68345. The government may have certain rights in the invention.

### FIELD OF THE INVENTION

The present invention relates to methods and compositions for enhancing an immune response in a mammalian subject. In particular, the present invention relates to antigen-polymeric delivery systems that comprise both an antigen and a basic additive.

### BACKGROUND

In recent decades, synthetic vaccines containing the epitopes for B-cells and/or T-cells have become one of the promising alternative approaches to the traditional vigorous vaccines in vaccine delivery and offer great advantages as far as safety is concerned. However, most synthetic peptide vaccines are poorly immunogenic and unable to elicit effective immune responses when used alone, which requires the co-administration of an adjuvant with low toxicity to enhance the immune responses. The only class of adjuvants that has been approved for human use in the US are aluminum salts. However, this class of adjuvants (aluminum salts) do not work well with low molecular weight small peptides due to the low adsorptive affinity of peptides onto the aluminum salts.

Vaccines against human chorionic Gonadotropin (hCG) are one of the most advanced contraceptive vaccines, which utilize the body's own immune defense system to provide protection against an unplanned pregnancy. The C-terminal peptide (CTP) portion of the beta chain of hCG has been previously studied as an immunogen because of its unique structure, unlike the alpha chain and the rest of beta chain that share common sequences with other hormones (such as hLH).[1] CTP antigens consisting of 35-37 residues were found to induce antibody responses that neutralize the biological activities of hCG but were not reactive toward hLH.[2] As synthetic peptide vaccines, which are chemically synthesized and purified, CTP antigens have the advantages of being safe relatively stable, easy and inexpensive to produce.[3] Synthetic vaccines containing the epitopes for B-cells and/or T-cells have become one of the promising alternatives approach to the traditional vigorous vaccines in vaccine delivery and offer great advantages as far safety is concerned. However, like most of the synthetic peptide vaccines, the CTP antigen are poorly immunogenic and unable to elicit effective immune responses when used alone, which requires the co-administration of an adjuvant with low toxicity to enhance the immune responses.

Most of currently studied hCG vaccines comprise covalently linked macromolecules such as diphtheria (DT) or tetanus toxoid (TT) as carriers to the immunogen to provide the T-cell helper effect in order to induce antibody response. Despite the advantages of being able to activate T-cell in a high population of humans without causing serious side effects and being reasonably inexpensive, these carriers have potential problems for long-term use, mainly limited by the need to provide T-cell help without MHC-restriction in order to produce high level of antibody response in virtually 100% of recipients.[4] Several “promiscuous” or universal T-cell epitopes that are not MHC restricted and thus broadly reactive in multiple haplotypes, have been identified and incorporation into synthetic antigens has been was rationally designed.[5, 6] The antibody levels induced by the synthetic peptide containing CTP and a universal T-cell epitope (TT2) were comparable to those elicited by the same peptide conjugated to DT.[4] Yet strong adjuvants or vehicles such as squalene, MDP or Arlacel A were still necessary to induce a sufficiently strong immune response. In addition, multiple administrations including 2-3 booster immunizations were required in order to maintain the antibody level long enough (months) to be useful as a vaccine.

The potential application of poly(lactide-co-glycolide) (PLGA) delivery systems in vaccination has been extensively investigated in recent decades.[7] Besides the biodegradable nature low immunogenicity and toxicity of this polymeric carrier, the prolonged antigen release pattern is one of the most attractive features for the development of single-shot vaccine formulations based on PLGA. Antigens encapsulated in PLGA microparticles slowly and continuously release out in vivo and stimulate lymphocytes including antigen presenting cells, which eliminate the need for multiple immunizations.[8-10] Besides providing prolonged antigen release, another appealing feature of PLGA microparticles as vaccine preparation has been discovered recently: that is, the adjuvancy of PLGA particles, especially their ability to elicit cellular immune responses in addition to producing antibodies in contrast to only FDA-approved adjuvant in human use-alum particles.[11, 12] Microparticles (<10 μm) were further shown to be able to become internalized by macrophages and stimulate certain CTL in vitro (Men et al. 1999), which is consistent with recent findings that particulate nature of antigens may be responsible for CTL responses.[13]

### SUMMARY OF THE INVENTION

The present invention provides polymeric delivery systems and methods of enhancing an immunogenic response in a subject. The new methods of enhancing an immunogenic response in a mammalian subject comprise administering a biodegradable polymeric delivery system comprising a biologically effective amount of one or more antigens and one or more basic additives to the mammalian subject. Preferably, the mammalian subject is a human subject. The antigen is selected from the group consisting of nucleic acids, proteins, polypeptides, peptides, polysaccharides, hapten conjugates, and combinations thereof. In a preferred embodiment, the antigen used in accordance with this method is a peptide.

The basic additive used in accordance with this method may be characterized by having a pH of a saturated solution at 37° C. in the range from about 6.8 to about 12.5 and a solubility in water at 37° C. from 1.2×10⁻² to about 3×10⁻¹¹. Especially suitable basic additives may be selected from the group consisting of magnesium carbonate, magnesium hydroxide, magnesium oxide, magnesium trisilicate, zinc carbonate, zinc hydroxide, zinc phosphate, aluminum hydroxide, basic aluminum carbonate, dihydroxyaluminum sodium carbonate, dihydroxyaluminum aminoacetate, ammonium phosphate, calcium phosphate, calcium hydroxide, magaldrate, calcium sulfate and combinations thereof. Other suitable basic additives may be used as well.

Also provided are methods of enhancing an immunogenic response to human chorionic gonadatropin (hCG) in a human subject, the method comprising administering a biodegradable polymeric delivery system comprising a biologically effective amount of an hCG antigen and a basic additive to the human subject. Preferably the hCG antigen is a carboxyl terminal peptide (CTP) of the beta subunit of hCG. In accordance with the present invention, the hCG can be conjugated to the polymeric delivery system, encapsulated in the polymeric delivery system, or both. Preferably, the polymeric delivery system comprises from 0.08 to 20% antigen based on the weight of the polymer.

The basic additive of the present invention may be characterized by having a pH of a saturated solution at 37° C. in the range from about 6.8 to about 12.5 and a solubility in water at 37° C. from 1.2×10⁻² to about 3×10⁻¹¹. The basic additive may be selected from the group consisting of magnesium carbonate, magnesium hydroxide, magnesium oxide, magnesium trisilicate, zinc carbonate, zinc hydroxide, zinc phosphate, aluminum hydroxide, basic aluminum carbonate, dihydroxyaluminum sodium carbonate, dihydroxyaluminum aminoacetate, ammonium phosphate, calcium phosphate, calcium hydroxide, magaldrate, calcium sulfate and combinations thereof, or another basic additive as determined by one of ordinary skill in the art. In a preferred embodiment, the basic additive is MgCO₃.

In accordance with the present invention, the ratio of basic additive to antigen may range from 0.5:1 to 30:1 (w/w). Preferably ratio of basic additive to antigen is about 4:1 (w/w). The ratio of basic additive to biodegradable polymer may be from 0.5 to 20% (w/w). Preferably, the ratio of basic additive to biodegradable polymer is from 1 to 7%. More preferably, the basic additive is added at a level of 3% or less based on the weight of the polymer.

The biodegradable polymeric delivery system is may be any suitable polymeric delivery system. One especially suitable polymeric delivery system is a poly(lactide-co-glycolide) (PLGA) delivery system. One preferred PLGA polymer is poly(D-L-lactide-co-glycolide). In accordance with the present invention, the ratio of lactide/lactic acid to the ratio of glycolide/glycolic acid is in the range from 100:0 to 0:100. Preferably, the ratio of lactide/lactic acid to the ratio of glycolide/glycolic acid is in the range from 100:0 to 50:50. The PLGA polymeric delivery system may further comprise an adjuvant and/or an excipient.

Also provided is an immunogenic composition for eliciting an immune response against an antigen comprising: (a) a biodegradable polymeric delivery system; (b) a biologically effective amount of an antigen; and (c) a basic additive.

In a preferred embodiment, the immunogenic composition is an immunogenic composition for eliciting an immune response against human chorionic gonadatropin (hCG) comprising: (a) a poly(lactide-co-glycolide) polymeric delivery system; wherein the ratio of lactide/lactic acid to the ratio of glycolide/glycolic acid is in the range from 100:0 to 50:50; (b) 0.08 to 20% (w/w) of an hCG antigen, based on the weight of the polymer, wherein the hCG antigen is a carboxyl terminal peptide (CTP) of the beta subunit of hCG; and (c) 0.5 to 20% (w/w) of a basic additive, based on the weight of the polymer, wherein the polymer is selected from the group consisting of magnesium carbonate, magnesium hydroxide, magnesium oxide, magnesium trisilicate, zinc carbonate, zinc hydroxide, zinc phosphate, aluminum hydroxide, basic aluminum carbonate, dihydroxyaluminum sodium carbonate, dihydroxyaluminum aminoacetate, ammonium phosphate, calcium phosphate, calcium hydroxide, magaldrate, calcium sulfate and combinations thereof.

### BRIEF DESCRIPTION OF THE FIGURES

FIG. 1. Scanning electron micrograph of peptide-conjugated (left panel), and -encapsulated (right panel) microspheres.

### DETAILED DESCRIPTION OF THE INVENTION

The present invention provides new biodegradable polymeric delivery systems that have one or more antigens encapsulated therein. The new systems also comprise one or more select basic additives or antacids encapsulated therein. The present systems are based, at least in part, on the discovery that PLGA microspheres which contain both an antacid and a peptide antigen produce a greater immunogenic response when injected into an animal than PLGA microspheres which contain the peptide antigen but lack the basic additive.

Definitions

The terms “polylactide” and “PLGA” as used herein are used interchangeably and are intended to refer to a polymer of lactic acid alone, a polymer of glycolic acid alone, a mixture of such polymers, a copolymer of glycolic acid and lactic acid, a mixture of such copolymers, or a mixture of such polymers and copolymers. A preferred polymer matrix for formation of the microspheres of the instant invention is poly(D-L-lactide-co-glycolide).

The term “antigen” as used herein denotes a compound containing one or more epitopes against which an immune response is desired. Typical antigens will include nucleic acids, proteins, polypeptides, peptides, polysaccharides, and hapten conjugates. Complex mixtures of antigens are also included in this definition, such as whole killed cells, bacteria, or viruses, or fractions thereof. In a preferred embodiment, the antigen is a peptide.

The term “biologically effective amount” as used herein denotes an amount of basic additive that enhances the immunogenic response of an immunized animal to the antigen.

The term “encapsulation” as used herein denotes a method for formulating an the antigen into a composition useful for controlled release of the antigen. Examples of encapsulating materials useful in the instant invention include polymers or copolymers of lactic and glycolic acids, or mixtures of such polymers and/or copolymers, commonly referred to as “polylactides” or “PLGA”, although any polyester or other encapsulating material may be used. The term “coencapsulation” as used herein refers to the incorporation of one or more antigens and one or more basic additives into the same polymeric delivery system.

The term “organic solvent” as used herein is intended to mean any solvent containing carbon compounds. Exemplary organic solvents include halogenated hydrocarbons, ethers, esters, alcohols and ketones, such as, for example, methylene chloride, ethyl acetate, a mixture of ethyl acetate and benzyl alcohol or acetone, dimethyl sulfoxide, tetrahydrofuran, dimethylformamide, and ethanol.

Polypeptide” as used herein refers generally to peptides and proteins having at least about two amino acids.

“Vaccine” as used herein refers to a formulation of an antigen intended to provide a prophylactic or therapeutic response in a host when the host is challenged with the antigen. Exemplary vaccines include vaccines directed against hCG.

Polymeric Systems

Two injectable polymer configurations are currently used to deliver peptides and proteins: spherical particles on the micrometer scale (˜0.5-2 μm), which are commonly referred to as “microspheres,” and single cylindrical implants on the millimeter scale (˜0.8-1.5 mm in diameter), which we term “millicylinders.” Both configurations are prepared from the biocompatible copolymer class, poly(lactide-co-glycolide) (PLGA) commonly used in resorbable sutures, and each configuration has distinct advantages and disadvantages (8).

Once injected into the body, these polymer implants slowly release the biologically active agents, thereby providing desirable levels of the agent over a prolonged period of time. Because of its safety, FDA approval and biodegradability, the poly(lactide-co-glycolides) (PLGAs) are the most common polymer class used for preparing biodegradable delivery systems for biologically active agents.

Lactide/Lactic Acid to Glycolide/Glycolic Acid Ratio

In accordance with the present invention, the polymeric delivery system of the present invention comprises polymers and co-polymers of lactide, lactic acid, glycolide, and glycolic acid (hereinafter referred to as “PLGA”). Virtually any ratio of lactide/lactic acid to glycolide/glycolic acid may be used, though it is preferred that the polymers of the present invention are in the ranges from 100:0 lactide/lactic acid:glycolide/glycolic acid to 50:50 lactide/lactic acid:glycolide/glycolic acid. It will be recognized by those of skill in the art that changing the ratio of lactide/lactic acid to glycolide/glycolic acid will affect the rate of biodegradation of the delivery system, and thus the release of the antigen to the subject. Accordingly, the appropriate ratio for particular antigens may readily be determined by those skilled in the art.

Size and Shape of the Delivery System

The delivery system of the present invention comprises micro- and nanoparticles, particularly microspheres, nanospheres, millicylinders, and the like. For convenience, all of these particles are hereinafter referred to generically as “microparticles.” The particles may be categorized into small, with diameters on the order of about 50 nm to about 500 nanometers; medium, with diameters in the range of about 500 nanometers to 200 microns; large, with diameters from about 200 to 5000 microns, and extra large, with diameters from about 5 millimeters to about 500 mm. Preferably, the microparticle has a diameter of 1-20 μm.

The antigen, preferably, is incorporated into the microparticle directly, during preparation of the microparticles. Although less preferred, the antigen, alternatively, may be conjugated to the outside of the microparticle. The delivery system may therefore contain microparticles with the antigen incorporated, microparticles with the antigen conjugated, or combinations of both. The amount of antigen used will depend on the antigen itself, it's solubility, it's predicted and actual loading efficiency, and so forth. The appropriate amount of antigen to encapsulate in or conjugate to the polymer can readily be determined by one skilled in the art. By way of example, the ratio of antigen to biodegradable polymer will generally be in the range from 0.1 to 20% (w/w). The actual antigen loading may be somewhat less based on the loading efficiency.

The “Antacid” or “Basic Additive”

In accordance with the present invention, an “antacid” or “basic additive” is incorporated into the delivery system along with the biologically active agent. The terms “antacid” and “basic additive” encompass compounds that counteract or neutralize acidity, such as alkalis or absorbents. Preferably, the “antacid” or “basic additive” will be a basic salt, wherein the pH of a saturated solution at 37° C. will be in the range of about 6.8 to about 12.5. For the purposes of the present invention, the antacids or basic additives preferably have a low solubility in water, wherein the solubility in water at 37° C. is in the range from about 1.2×10⁻² to about 3×10⁻¹¹. “Antacid” and “basic additive,” as used herein, are interchangeable. Some suitable basic additives include, but are not limited to, magnesium carbonate, magnesium hydroxide, magnesium oxide, magnesium trisilicate, zinc carbonate, zinc hydroxide, zinc phosphate, aluminum hydroxide, basic aluminum carbonate, dihydroxyaluminum sodium carbonate, dihydroxyaluminum aminoacetate, ammonium phosphate, calcium phosphate, calcium hydroxide, magaldrate, calcium sulfate and combinations thereof. Other suitable antacids or basic additives will be recognized by those of skill in the art.

Preparation of PLGA Microparticles

General Procedure for Preparation of the Microparticles In general, microencapsulation of the antigen is performed according to the any one of the several protocols that follow. Initially, PLGA of the desired ratio of lactide to glycolide (about 100:0 to 0:100 weight percent, more preferably, about 50:50 to 100:0, most preferably about 50:50) and inherent viscosity (generally about 0.1 to 1.2 dL/g, preferably about 0.2 to 0.8 dL/g) is first dissolved in an organic solvent such as methylene chloride, or ethyl acetate with or without benzyl alcohol or acetone to the desired concentration (generally about 0.05 to 1.0 g/mL, preferably about 0.2 to 0.8 g/mL). A concentrated antigen solution (for example, typically at least 0.1 mg/mL for polypeptides, preferably greater than about 100 mg/mL, depending, for example, on the type of polypeptide and the desired core loading). Dry antigen may be used in place of aqueous antigen. The antacid is also introduced into the solvent, before, after, or contemporaneously with the polymer. The ratio (w/w) of antacid to polymer in this initial formulation is from about 0.5 to 20%, preferably from 1 to 7%. As shown in the examples below, particularly good results have been achieved using 3% by weight of antacid to polymer. The ratio (w/w) of antacid to antigen is from 0.5:1 to 30:1. As shown in the examples below, particularly good results have been achieved using a ratio of approximately 4 parts (w/w) of antacid to 1 part of antigen. The amount of protein or peptide incorporated into the formulation preferably is from 0.08 to 20% (w/w) of the polymer.

Any one of a number of methods know in the art may be employed to produce the present microparticles. One exemplary method utilizes a solvent evaporation technique. A solid or liquid active agent is added to an organic solvent containing the polymer. The active agent is then emulsified in the organic solvent. This emulsion is then sprayed onto a surface to create microparticles and the residual organic solvent is removed under vacuum. Another exemplary method involves a phase-separation process, often referred to as coacervation. A first emulsion of aqueous or solid active agent dispersed in organic solvent containing the polymer is added to a solution of non-solvent, usually silicone oil. By employing solvents that do not dissolve the polymer (non-solvents) but extract the organic solvent used to dissolve the polymer (e.g. methylene chloride or ethyl acetate), the polymer then precipitates out of solution and will form microparticles if the process occurs while mixing. A third exemplary method utilizes a coating technique. A first emulsion comprising the active agent dispersed in a organic solvent with the polymer is processed through an air-suspension coater apparatus resulting in the final microparticles.

The microparticles of the instant invention are preferably formed by a water-in-oil-in-water emulsion process. Additional examples of these and other suitable methods for preparing the microparticles are described below.

Preparation of PLGA 50/50 (0.64 dl/g) Microparticles A solution of the antigen of interest (150 mg/mL or 300 mg/mL) in 10 mM phosphate buffer (pH 7.4) is added to 1 mL of 30% w/v PLGA-CH₂Cl₂ solution containing the antacid or basic salt. The mixture is then homogenized at 10,000 rpm (Homogenizer: Model IQ², VirTis Co., Gardiner, N.Y.) for 1 minute, in an ice bath. The formed water-in-oil (W/O) emulsion is added immediately to 1 mL of 2% w/v polyvinyl alcohol (PVA) aqueous solution, and the mixture is vortexed for 20 seconds to form a water-in-oil-in-water (W/O/W) double emulsion. The double emulsion is immediately transferred to 100 mL of 0.5% w/v PVA, aqueous solution, under stirring, at a constant rate. The microspheres are stirred continuously for 3 hours at room temperature. The hardened microspheres are collected by centrifugation and washed with ice-cold water 3 times. Finally, the microspheres are lyophilized for 24 hours to get the final dry product using a Labcono FreeZone® 6 Liter Freeze Dry System (Kansas City, Mo.).

Preparation of PLGA 50/50 (0.20 dl/g) Microparticles All the materials and procedures are the same except that 70% w/v polymer concentration is used instead of 30%.

Preparation of PLGA Microparticles by the Oil-in-oil (O/O) Emulsion Method/Solvent Extraction Method The antigen (directly ground from the lyophilized powder and sieved to <45 μm) is added to the polymer solution in 1 mL of acetonitrile. The suspension is homogenized at 15,000 rpm (Model IQ² homogenizer, Virtis Co., Gardiner, N.Y.) for 3 minutes on an ice bath, and then is slowly added, dropwise, to 100 mL of cotton seed oil (Sigma Chemical Co.) containing 1.6 grams of Span 85, under stirring at 700 rpm. The formed O/O emulsion is then stirred continuously under ambient conditions for 5 hours. Thereafter, 100 mL of petroleum ether is added, and stirring is continued for another 15 minutes. The microparticles are then collected by filtration through a 0.45 μm membrane filter (Gelman Sciences) and are lyophilized at room temperature for 2 days.

Preparation of Antigen/PLGA Microparticles by the W/O/W Emulsion Method To reduce the burst effect, generally, the volume ratio of the internal phase (protein solution) to the external phase (polymer solution) should be below 1:10, and higher polymer concentration should be used [Cleland 1997]. Therefore, the ratio of 1:10, and the PLGA 50/50 (0.64 dl/g) concentration of 300 mg/mL (700 mg/mL for PLGA50/50 (0.20 dl/g)) is used for all the preparations, which results in high encapsulation efficiency for these preparations (i.e., >80%). By SEM, PLGA microspheres prepared by this method appear mostly spherical with very smooth surfaces and their sizes range from about 60 to about 70 μm.

The microparticles of the instant invention may be prepared to any desired size by varying process parameters such as stir speed, volume of solvent used in the second emulsion step, temperature, concentration of PLGA, and inherent viscosity of the PLGA polymers.

Antigens

Although any antigen, as defined above, may be incorporated into the polymeric delivery vehicle, it is expected that the antigen or interest will be a protein or polypeptide. Polypeptides or protein fragments defining immune epitopes, and amino acid variants of proteins, polypeptides, or peptides, may be used in place of full length proteins. Polypeptides and peptides may also be conjugated to haptens. Polypeptides which comprise both a B cell epitope and a T cell epitope, particularly a universal or “promiscuous” helper T cell epitope, i.e, a T cell epitope which is not MHC restricted, are particularly useful. Other useful polypeptides are multivalent polypeptides which comprise both a B cell epitope and a cytotoxic T cell epitope.

Typically, an antigen of interest will be formulated in PLGA microparticles to provide a desired period of time between the first and second bursts of antigen and to provide a desired amount of antigen in each burst. Microparticles containing antigen and the basic additive may be formulated to release adjuvant in a pulsatile manner or to continuously release adjuvant.

The PLGA microparticles comprising encapsulated antigen and basic additive may be used alone or in any combination with soluble antigen, or with microparticles which comprise an antigen that is conjugated to the microparticle. Methods for preparing microparticles for conjugated proteins are described in U.S. Pat. No. 6,326,021, issued Dec. 4, 2001, which are specifically incorporated herein by reference. The microparticles are placed into pharmaceutically acceptable, sterile, isotonic formulations together with any required cofactors, and optionally are administered by standard means well known in the field. Microparticle formulations are typically stored as a dry powder.

It is envisioned that injections (intramuscular or subcutaneous) will be the primary route for therapeutic administration of the microparticles of this invention, although intravenous delivery, or delivery through catheter or other surgical tubing is also used. Alternative routes include suspensions, tablets, capsules and the like for oral administration, commercially available nebulizers for liquid formulations, and inhalation of lyophilized or aerosolized microcapsules, and suppositories for rectal or vaginal administration. Liquid formulations may be utilized after reconstitution from powder formulations.

The adequacy of the vaccination parameters chosen, e.g. dose, schedule, and the like may be determined by taking aliquots of serum from the patient and assaying antibody titers during the course of the immunization program. Alternatively, the presence of T cells or other cells of the immune system may be monitored by conventional methods. In addition, the clinical condition of the patient may be monitored for the desired effect, e.g. anti-infective effect. If inadequate vaccination is achieved then the patient may be boosted with further vaccinations and the vaccination parameters may be modified in a fashion expected to potentiate the immune response, e.g. increase the amount of antigen, complex the antigen with a carrier or conjugate it to an immunogenic protein, or vary the route of administration.

The degradation rate for the microparticles of the invention is determined in part by the ratio of lactide to glycolide in the polymer and the molecular weight of the polymer. Polymers of different molecular weights (or inherent viscosities) may be mixed to yield a desired pulsatile degradation profile. Furthermore, populations of microparticles designed to have the second burst occur at different times may be mixed together to provide multiple challenges with the antigen at desired intervals. Similarly, mixtures of antigens may be provided either together in the same microparticles or as mixtures of microparticles to provide multivalent or combination vaccines. Thus, for example, rather than receive three immunizations with traditional vaccine at 2, 4, and 6 months, a single microencapsulated vaccine may be provided with microparticles that provide second bursts at 2, 4, and 6 months.

Further details of the invention may be found in the following examples, which further define the scope of the invention. All references cited herein are expressly incorporated by reference in their entirety.

### EXAMPLE 1

MATERIALS AND METHODS Poly(D,L-lactide-co-glycolide) 50/50, end-group capped, with an inherent viscosity of 0.19 dl/g in HFIP at 30° C. was obtained from Birmingham Polymers, Inc. (Birmingham, Ala.). Poly(L-lysine) hydrobromide (MW 150-300 kDa), MgCO₃, L-ornithine hydrochloride, and 5,5′ dithio-bis (2-nitrobenzoic acid) were purchased from Sigma Chemical Co. (St. Louis, Mo.). Poly(vinyl alcohol) (80% hydrolyzed, MW 9-10 kDa) was obtained from Aldrich Chemical Co. (St. Louis, Mo.). N-3-maleimido-butyryloxysulfosuccinimide ester and Coomassie Plus assay kit were purchased from Pierce. All other reagents were analytical grade or higher and used as received.

CTP37-TT2 antigen The synthetic human chorionic gonadotropin (hCG) peptide antigen consists of a B-cell epitope from C-terminal portion of beta chain of hCG (residues 109-145) and a universal or “promiscuous” T-cell epitope from tetanus toxoid (residues 830-844, designated as TT2), which are co-synthesized and separated by a spacer. The amino acid sequence of this peptide is: C-QYIKANSKFIGITEL (TT2)-DDPRFQDSSSSKAPPPSLPS—PSRLPGPSDTPILPQ (βhCG (109-145), also CTP37). A cysteine residue was inserted at one end of the sequence to make it convenient for further conjugation vial thiol group without altering B- and T-cell epitopes. This synthetic immunogen was able to elicited antibody responses comparable to those induced by the same βhCG peptide conjugated to diphtheria toxoid (DT).

Encapsulation of CTP37-TT2 antigen in PLGA microspheres The peptide was encapsulated in PLGA microspheres by a double emulsion-solvent evaporation (W/O/W) method with an antacid (i.e., MgCO₃) suspended in the PLGA matrix. Briefly, PLGA was dissolved in methylene chloride at a concentration of 700 mg/mL. 3% (w/w, MgCO₃:PLGA) MgCO₃ pre-sieved through 45 μm US standard steel sieve was suspended uniformly in the polymer solution. 100 μl of 70 mg/mL peptide in PBS solution were added and the mixture was homogenized at 15,000 rpm for 1 minute over an ice bath to form a w/o emulsion. To this primary emulsion, 2 mL of 5% (w/v) PVA solution (80% hydrolyzed, MW 9-10 kDa) was added and further homogenized at 10,000 rpm for 1 minute. The formed w/o/w emulsion was in-liquid hardened in a large volume of PBS containing 0.5% (w/v) PVA for 2 hours under stirring. The microspheres were collected by centrifugation, washed, and lyophilized. Blank microspheres, either with or without MgCO₃ incorporated in the polymer matrix, were prepared in the same manner, except that no peptide was present.

Preparation of PLGA microspheres with surface conjugatable groups (PLGA/pLys microspheres) The PLGA microspheres surface-modified with polylysine were prepared similarly as described previously, except that higher homogenization speed was used in order to produce smaller particles. Briefly, PLGA was dissolved in methylene chloride at a concentration of 500 mg/mL. The dissociation degree of polylysine in water solution (5.0 mg/mL) was adjusted to 85% with 1 N NaOH prior to microspheres preparation. 1.25 mL of the polylysine solution was then added to 0.25 mL of the PLGA/CH₂Cl₂ solution and the mixture was homogenized at 15,000 rpm for 1 minute. The resultant emulsion was hardened in 100 mL of distilled water for 3 hours under stirring. The microparticles were collected by centrifugation following sieving through 45 μm US standard steel sieve and washed 3 times with 0.15 N NaCl, freeze-dried in ˜0.04 N NaCl and 7.5% sucrose. The content of polylysine entrapped in the microspheres was determined by a pre-column derivatization RP-HPLC method as described earlier by Cui.

Preparation of CTP37-TT2 peptide for conjugation. The peptide was first dissolved in 0.1 M sodium phosphate buffer, (pH 8.0) or a buffer solution containing 0.05 M sodium phosphate, 0.1 M NaCl and 6 M Guanidine.HCl (pH 7.4), at a concentration of 5-20 mg/mL. DTT was then added to a final concentration of 100-300 mM and the mixture was incubated at room temperature for 2-4 hours. The excess reagent was removed either by dialyzing with Slide-A-Lyzer dialysis cassettes (Pierce Chem. Co, MWCO=2000 Da) at 4° C. against a buffer containing 0.05 M sodium phosphate, 0.1 M NaCl and 0.01 M EDTA (pH 7.4) or by passing the solution through PD-10 column twice, which was equilibrated and eluted with the same buffer. The free thiol groups exposed after reduction were determined by 5,5′ dithio-bis (2-nitrobenzoic acid) (Elman's reagent). The pH of the reduced peptide solution was adjusted to 6.6 prior to conjugation.

Conjugation of the peptide to PLGA/pLys microspheres. The PLGA/pLys microspheres were suspended in 0.1 M sodium phosphate buffer (pH 6.6) after sucrose was removed by washing for three times. N-3-maleimido-butyryloxysulfosuccinimide ester (Sulfo-GMBS) was added slowly to the suspension under stirring at 4:1 molar excess, equivalent to 60 nmol/mg microspheres, to the lysine residues on the microspheres. The mixture was allowed to stand for 1-3 hours in the dark. Following removal of the excess reagent by washing for several times, the microspheres were re-suspended in the same buffer. Aliquots of the suspension were taken to determine contents of the maleimido groups introduced by sulfo-GMBS in the microspheres, by assaying the capacity of cysteine consumability with Elman's reagent. An amount of reduced peptide equivalent to 1.2 molar excess of the determined maleimido groups was added to the microsphere suspension and the reaction was continued for 2 hours in the dark. The microspheres were washed with H₂O followed by freeze-drying in 10% (w/v) sucrose solution. The content of polymer microspheres in the formulation was determined by weighing the dried particles before and after washing off sucrose. The peptide-conjugated microsphere formulation was washed with water, PBS, or 2% (w/v) SDS solution prior to determination of the peptide loading (see below).

Determination of the peptide loading. The antigen encapsulated in microspheres was simply determined by Coomassie Plus assay (Pierce Chem. Co.) after extracting the peptide from the microspheres. The peptide-encapsulated microspheres were suspended in acetone and the dissolved polymer was removed by centrifugation. The precipitated peptide pellet was washed twice and acetone was allowed to evaporate before the peptide pellet was reconstituted in PBS solution. The Coomassie assay was then performed in these aqueous samples.

The amount of the peptide conjugated to the microsphere was determined by a pre-column OPA derivatization RP-HPLC assay. The methods of preparation of reagents and sample derivatization were as previously described by Cui except L-omithine was used as the internal standard. Standards were prepared using pure polymer and a series of known amount of peptide and following the same procedures as samples. The method was validated before assay of microspheres formulations. Around 3 mg of dry microspheres, salt and sucrose-free, or pure polymer and 7 mmol of L-omithine (I.S.) were completely hydrolyzed in 6 N HCl at 110° C. under light vacuum for 22 hours. After removal of hydrochloric acid, the hydrolyzed amino acids were reconstituted in 1 M sodium carbonate solution (pH 9.5), derivatized with OPA/2-ME reagent, and injected 120 μl into an ODS column (Nova-Pak C18, 3.9×150 mm, 4 μm, Waters, Milford, Mass.). A binary gradient mobile phase consisting of 0.05 M sodium acetate buffer, pH 6.8 (eluent A) and 100% methanol (eluent B) was used. The flow rate was 1.5 mL/minute over 22 minutes. A gradient program was followed: 49% methanol in 0-7.5 minutes, 49-65% up to 12 minutes, 65% between 12-15 minutes, and 65-49% up to 16 minutes.

SEM Image Analysis of PLGA Microspheres The morphology of the microparticles was analyzed with a scanning electron microscope (Philips XL-30). Microparticles were freeze-dried after removal of salts and sucrose in the sample before coating with a thin gold layer in an argon atmosphere using a Pelco Model 3 sputter coater for SEM use.

FITC-Labeling of CTP37-TT2 and Laser Confocal Scanning Microscopy 400 μg of FITC was dissolved in 100 mM carbonate buffer (pH 9.0) and added immediately to 2 mL of 1 mg/mL CTP37-TT2 in the same buffer solution. The mixture was incubated at 37° C. for 1 hour in the dark. The FITC-labeled peptide was purified by dialysis and lyophilized. The peptide was conjugated to PLGA microspheres as described above (sections Preparation of CTP37-TT2 peptide for conjugation & Conjugation of the peptide to PLGA/pLys microspheres). The distribution of the FITC-(CTP37-TT2) in the dry PLGA microspheres was analyzed using a Zeiss laser confocal scanning microscope (LCSM). The excitation wavelength, 488 nm, was provided by a Argon laser and a 63× objective was used for magnification. The pinhole was set at <2.2 μm. Median cross-sections of the microspheres were examined.

In vitro release studies Around 15 mg of peptide-conjugated microspheres were incubated in 0.5 mL of PBS containing 0.02% Tween 80 (PBST) at 37° C. after washing off sucrose with the same buffer for twice. For peptide-encapsulated microspheres, 30-35 mg were incubated in 0.5 mL of PBST likewise. The supernatant solution was removed at different time intervals, i.e., 1, 3, 7, 11, 14, 25, 35 days. For the peptide-conjugated formulation, the peptide released in the release media was determined by RP-HPLC method following acid hydrolysis as described in Determination of peptide loading section. The peptide released from encapsulated formulation was determined by Coomassie and RP-HPLC assay.

Immunization Protocol. The immunogenicity of CTP37-TT2 antigen, surface-conjugated or -encapsulated in PLGA microspheres, was tested in rabbits. Six groups (Group I-VI) of five rabbits were administered intramuscularly with microspheres formulations following the immunization scheme as described in Table 2. All microsphere formulations were suspended in 1 mL PBS just prior to injection. One dose of 1.0 mg soluble peptide in PBS solution was injected as a negative control (group VII). A synthetic analogue of a surface component of mycobateria, N-acetyl-nor-muramyl-L-alanyl-D-isoglutamine (nor-MDP), encapsulated in a separate PLGA microsphere formulation, was co-administered in groups IV-VII. Peptide associated with a w/o emulsion, i.e., PBS (containing nor-MDP) in squalene:mannide monooleate (4:1) emulsion (water/oil phase volume ratio=40:60), served as a positive control (Group VIII) and was boosted at 4 & 10 weeks. Blood samples were collected weekly between 2 weeks and 6 months. The serum antibody binding to iodine-labeled hCG was determined by radioimmunoassay (RIA) [16]. The tissue of the rabbits at the site of injection was examined at the end time point of the in vivo study and the severeness of tissue reactions (such as inflammation, formation of granulomous) was scored from 0 to 5.

Results And Discussion

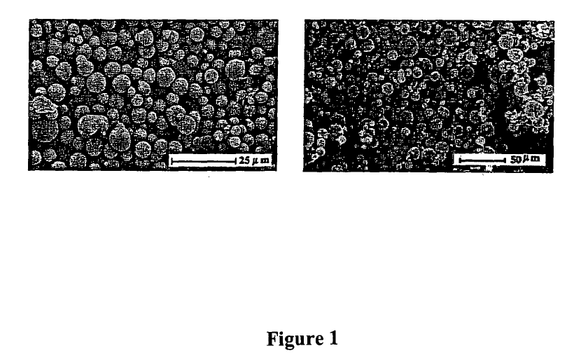

CTP37-TT2 peptide-encapsulated PLGA microspheres The CTP37-TT2 antigen was encapsulated inside PLGA microspheres in order to provide a prolonged release (>1 month) of the immunogen and eliminate the need of booster immunization. In addition, to interact with and target to antigen presenting cells, a small particle size (1-10 μm) was desired. The microspheres were prepared with a commonly used double emulsion (w/o/w)-solvent evaporation method. The loading of the peptide was limited by the solubility of this peptide (˜70 mg/mL) and the theoretical loading was of 1% in the microspheres. Microspheres containing 0.63-0.76% (w/w) antigen, with a mean particle size of 3.8 μm were obtained (Table 1). The encapsulation efficiency was around 63-76%, varied slightly within batches. The microspheres were spherical in shape and with smooth surface (FIG. 1).

An acidic microclimate pH has been shown to occur in PLGA microspheres during polymer incubation and degradation at 37° C., which has a detrimental effect on the stability and structural integrity of encapsulated proteins. Co-encapsulation of insoluble antacid or bases, such as MgCO₃, in PLGA implants and microspheres was found to counteract the acidic microclimate pH and stabilize acid-induced protein instability.[17, 18] To inhibit the potential deterious effects of the microclimate acidity on the CTP37-TT2 antigen, MgCO₃ was incorporated in the polymer matrix during microsphere preparation. The inorganic base was sieved through 45 μm sieve before use. Taking into account that the microsphere product has such a small particle size, the extent to which MgCO₃ powder was encapsulated into the particles, was questionable. Observation of MgCO₃ suspended in PLGA/CH₂Cl₂ solution under microscope revealed the majority of the powder fell in the size of 2-24 μm, irrespective of the presence of a small fraction of big particles. Flame Atomic spectroscopy of blank PLGA microspheres prepared under the same condition but without addition of the antigen showed around 36% of MgCO₃ was actually incorporated in these small particles. Therefore, it was likely that appreciable amount of MgCO₃ (˜36%) was suspended in the PLGA microspheres encapsulating the antigen.

The effect of MgCO₃ on the stability of the peptide encapsulated during in vitro release was investigated (paper in preparation). It was found that within 1-month release, incorporation of MgCO₃ has no significant effect on the peptide stability in PLGA microspheres. Another interesting founding was that MgCO₃ may play an important role on the immunogenicity of antigen-encapsulated PLGA microspheres (see below).

Conjugation of CTP37-TT2 peptide antigen on PLGA/pLys microsphere surface Protein or peptide antigens covalently linked to microspheres surface have shown to induce strong CD4⁺ T cell responses and elicit good antibody production in mice and monkeys.[14, 15] The presence of antigen on the surface of particles was speculated to be important in phagocytosis of the particulate antigens and subsequent induction of immune responses. Conjugation of CTP37-TT2 peptide antigen to biodegradable PLGA microspheres became possible with the production of PLGA microspheres with surface-conjugatable moities. The immunogenicity of the surface-conjugataed antigen with or without combination of antigen depot in peptide-encapsulated micropspheres was examined in rabbits (see below).

PLGA microspheres with surface-conjugtable groups for further conjugation was prepared by one-step physical entrapment of polylysine as described. Smaller size of particles within 1-15 μm were preferred here for the same reason as mentioned above, that is, because of their capability of being phagocytosed by antigen presenting cells (APCs) and eliciting immune response. Using the modified preparation method, the PLGA/pLys microspheres with the mean particle size of 5.7±2.0 μm (N=115 SD) were produced. The polylysine content was 14.4±0.3 mol/mg (N=3±SD), which correspond to 0.30% of polylysine loading. The amino groups exposed on the PLGA/pLys microparticle surface after surface-entrapment of pLys was utilized for further covalently linking the peptide to the PLGA microparticle surface.

The conjugation was accomplished by a water-soluble bifunctional crosslinker (sulfo-GMBS, Pierce) and was performed in two steps, i.e., coupling of sulfo-GMBS to microsphere surface via reaction between primary amines and NHS ester of sulfo-GMBS, and subsequent coupling of the reduced peptide via reaction between the free thiol and maleimido groups [19]. For this purpose, the peptide was reduced before coupling. Direct and predictable conjugation of peptide on PLGA/pLys microsphere surface may be assured and no crosslinking was expected. With a spacer between the microsphere surface and peptide chain, the peptide was expected to be readily accessible on the surface. As shown in FIG. 1 and Table 1, about 0.8 μg of peptide was coupled to per mg of bulk microspheres. Washing with PBS buffer solution and 2% SDS solution did not alter the peptide loading to a significant extent as determined by RP-HPLC assay, which suggested that peptide are more likely covalently coupled than nonspecifically adsorbed onto microspheres. There was no effect of surface-conjugation of the peptide on the size (Table 1) and morphology of the microparticles (data not shown), spherical in shape and with smooth surface.

The distribution of the peptide following surface-conjugation was qualitatively determined by laser scanning confocal microscopy (LSCM), after the peptide was labeled with fluorescent probe (Fluorescein). The FITC-labeled peptide was conjugated to the PLGA/pLys microspheres in the same manner as unlabeled peptide. A strong fluorescence was observed dominantly from the surface of the particles, suggesting most of the peptide was associated with the microsphere surface. Washing the particles with 2% SDS obviously did not diminish the surface fluorescence, further illustrating that surface-associated peptide was more likely conjugated than non specifically adsorbed as concluded above. A certain extent of the distribution of the peptide in the interior of the particles, probably by adsorption and diffusion, was also observed.

In vitro release studies For both the peptide-encapsulated and -conjugated microspheres, the burst release of CTP37-TT2 antigen within 1 day was around 13% of the total peptide loading. Considering the peptide loading in encapsulated microspheres was around 10 times of that in the surface-conjugated formulation, the actual amount of peptide released from encapsulated microspheres was much higher. CTP37-TT2 in dilute solution was found unstable at neutral pH (paper in preparation), therefore, it was possible that the peptide released into the media for longer than 2-3 days aggregated and undetected so that release percentages were underestimated, especially in the later stage of release study (sampling intervals ˜10 days). Therefore, in this case, the release data were presented as a concentration profile of the peptide (ug peptide/mg microspheres). It was observed that the CTP37-TT2 peptide was slowly and continuously released from both formulations until ˜1 month. The peptide release detected from the encapsulated formulation during the later stage of the release was much lower than that within the first day. Whereas, in peptide-conjugated formulation, the peptide concentration at later time points where release media was collected were comparable to each other, which suggest appreciable amount of peptide released. However, due to the extremely low loading of conjugated peptide, the concentration (μg peptide/mg microspheres) remained low.

By summing up the peptide detected in the release media, a trend of continuous fast release in peptide-conjugated microspheres up to more than 60% after two weeks, followed by a slower release rate was observed. On the other hand, for the peptide-encapsulated microspheres, a more flattered release pattern was obtained. The concentration of the peptide in the release media was too low (except 1 day) to be detected by Coomassie assay. From RP-HPLC assay after complete hydrolysis of fractions of release media, only ˜5% more of peptide released until 25 days following bust release.

Immunogenicity of surface-conjugated and -encapsulated CTP37-TT2 antigen in rabbits. It was observed that all PLGA microsphere-associated CTP37-TT2 peptide (group I-VI) acted quickly and the Ab peak level was observed at ˜35-56 days, in contrast to the soluble peptide (˜77 days). Among these groups, the single-dose encapsulated CTP37-TT2 immunogen elicited an immune response much higher than that from the peptide administered in the w/o emulsion at multiple (3) doses in the positive control group (peak value 1250 vs. 400 nM). Co-administration of nor-MDP containing microspheres did not enhance, instead, decreased the antibody levels. Surface-conjugation of the peptide to PLGA microspheres also enhanced its immunogenicity at 1/5 of the dose relative to the soluble control group (peak value 40 vs. 20 nM). Co-administration of nor-MDP containing microspheres also decrease the antibody levels, while lengthened the duration of antibody response. Immunization with 1.0 mg encapsulated and 200 μg surface-conjugated peptide induced higher antibody responses than either administered alone. Combination of both encapsulated and conjugated peptide plus nor-MDP adjuvant as in group VI induced an enormously high anti-hCG Ab response (peak value around 2800 nM). A prolonged duration of the high antibody level was observed in all microsphere formulations and was comparable to the multiple dose administration of positive control.

However, tissue examination showed that the high responders were accompanied with lumps and inflammation at the injection sites.

### EXAMPLE 1 CONCLUSIONS

The immunogenicity of CTP37-TT2 peptide antigen may be enhanced by encapsulation, or surface-conjugation, in PLGA microparticles. Combination of surface-conjugated and encapsulated CTP37-TT2 peptide antigen provided a long-lasting high anti-hCG antibody response after a single dose.

TABLE 1
Characteristics of the PLGA microspheres

| | Other | Peptide | Encapsulation | Particle | Burst Release |
|---|---|---|---|---|---|
| Formulation | Excipients | Loading | Efficiency | Size (μm)^{a} | (within 1 day) |
| Peptide-conjugated | | 0.084% | | 5.3 ± 0.1 | 12.5% |
| microspheres | | | | | |
| Peptide- | 3% MgCO₃ | 0.63-0.76% | 63-76% | 3.8 ± 0.3 | 13.3% |
| encapsulated | | | | | |
| microspheres | | | | | |

^{a}N = 115 + SEM

TABLE 2
Immunization Protocol to evaluate the immune responses elicited after i.m.
administration of several CTP37-TT2 vaccine formulations in rabbits.

| | | PLGA | | | |
|---|---|---|---|---|---|
| | Immunogen | Microspheres | Co-administered | Number of | Schedule |
| Group | (Dose) | administered (mg) | adjuvants (dose) | immunizations | (weeks) |
| I | SCF^{a} (200 μg) | 382 | None | 1× | 0 |
| II | EnF^{b} (1.0 mg) | 143 | None | 1× | 0 |
| III | SCF^{a} (200 μg) + | 525 | None | 1× | 0 |
| | EnF^{b} (1.0 mg) | | | | |
| IV | SCF^{a} (200 μg) | 382.625 | Nor-MDP^{e} (25 μg) | 1× | 0 |
| V | EnF^{b} (1.0 mg) | 143.625 | Nor-MDP^{e} (25 μg) | 1× | 0 |
| VI | SCF^{a} (200 μg) | 525.625 | Nor-MDP^{e} (25 μg) | 1× | 0 |
| VII | CTP37-TT2/PBS^{c} | 0.625 | Nor-MDP^{e} (25 μg) | 1× | 0 |
| | (1.0 mg) | | | | |
| VIII | CTP37-TT2/w/o | 0 | Nor-MDP^{f} (25 μg) | 3× | 0, 4, 10 |
| | emulsion^{d} (1.0 mg) | | | | |

^{a}Surface-conjugated formulation (SCF): PLGA microsphere formulation with the peptide conjugated on the surface.
^{b}Encapsulated formulation (EnF): PLGA microsphere formulation with the peptide encapsulated.
^{c}The soluble peptide in PBS solution was administered as a negative control.
^{d}The peptide was incorporated in a water-in-oil (PBS-in-squalene:mannide monooleate (4:1) (40:60)) emulsion.
^{e}nor-MDP was encapsulated in 0.624 mg PLGA microspheres.
^{f}nor-MDP solution was used.

TABLE 3
Tissue reaction at sacrifice after i.m. administration of several
CTP37-TT2 vaccine formulations in rabbits have a scoring system
from 0-3 with 0 being no pathology and 3 being severe pathology.
Any score above 0.5 is considered unacceptable for human use.

| Group^{a} | Score of tissue response | Time of sacrifice |
|---|---|---|
| I | 0.0 ± 0.0 | 24 weeks |
| II | 0.4 ± 0.2 | 24 weeks |
| III | 0.7 ± 0.4 | 24 weeks |
| IV | 0.0 ± 0.0 | 24 weeks |
| V | 0.5 ± 0.0 | 24 weeks |
| VI | 1.9 ± 0.2 | 24 weeks |
| VII | 0.0 ± 0.0 | 24 weeks |
| VIII | — | |
| Blank^{b} | 0.5 ± 0.0 | 4 weeks |
| Blank/MgCO₃^{c} | 0.9 ± 0.2 | 4 weeks |

^{a}Group I-VIII as described in Table 2.
^{b}Blank microspheres. Neither antigen nor MgCO₃ was encapsulated;
^{c}Blank microspheres. 3% MgCO₃ was added during microsphere preparation. No antigen was present.

### EXAMPLE 2

Purpose. To test the stability of a synthetic human chorionic gonadotropin antigen (CTP37-TT2), in the solution/solid-state and in poly(D,L-lactide-co-glycolide) (PLGA) microspheres for potential use as a birth control vaccine.

Methods. The stability of the non-encapsulated peptide was examined by monitoring the extent of peptide hydrolysis and insoluble aggregation at 37° C. in dilute solution (0.15-1 mg/mL) and in the solid state (97% RH) at a pH range of 2-7. The hCG antigen was encapsulated in PLGA (50/50, i.v. =0.20 dl/g) microspheres by the solvent evaporation method. The release kinetics of the peptide from the microspheres was monitored in PBS/0.02% Tween 80 at 37° C. Soluble peptide was detected by Coomassie® Plus protein assay and peptide integrity by SDS-PAGE. The total peptide retained in microspheres was evaluated by amino acid analysis.

Results. In solution, the peptide rapidly formed insoluble aggregates at pH 4-7. By one week, 65-75% of the peptide (1 mg/mL) became insoluble. The peptide was stable to hydrolysis except when the pH was very low (1-2). Sucrose, sorbitol, arginine and glycine (100:1 excipient/peptide, w/w) were found to significantly reduce the aggregation rate. In the solid state, the peptide was more stable. Only ˜10% aggregation after one week was recorded when lyophilized from pH 7 and reduced hydrolysis occurred in the acidic samples (pH 2). Continuous release of the hCG peptide from the microspheres of 1-15 μm was observed for over a month, with ˜70% remaining soluble and mostly unhydrolyzed.

Materials The amino acid sequence of CTP37-TT2, the synthetic human chorionic gonadotropin peptide antigen used in this study, is: CQYIKANSKFIGITELDDPRFQDSSSSKAPPPSLPSPSRLPGPSDTPILPQ, consisting of a B-cell epitope from C-terminal portion of beta chain of hCG (βhCG (109-145), also CTP37) and a universal or “promiscuous” T-cell epitope from tetanus toxoid (residues 830-844, designated as TT2). Poly(D,L-lactide-co-glycolide) 50/50, end-group capped, with an inherent viscosity of 0.19 dL/g in HFIP at 30° C. was obtained from Birmingham Polymers, Inc. (Birmingham, Ala.). MgCO₃ was purchased from Sigma Chemical Co. (St. Louis, Mo.) and sieved through 45 μm steel US standard sieve before use. O-phthalaldehyde was purchased from Alltech Associates Inc. (State College, Pa.). The Coomassie brilliant blue plus protein assay kit was purchased from Pierce Chemical Inc. All other reagents were analytical grade or higher and used as received.

Solution stability studies To evaluate the pH dependence of the stability of CTP37-TT2, the peptide was dissolved in a 10 mM sodium phosphate buffer (pH 7.4) at a concentration of 1.8 mg/mL. The solution was dialyzed against the same buffer, followed by water, at 4° C. with dialysis tubing of MWCO 1,000 Da. The dialyzed peptide solution was diluted with appropriate buffer to a concentration of 1 mg/mL and of various pHs (1.4, 2.2, 4.1 and 7.2). The final peptide solution consists of either 10 mM citrate/HCl buffer for pH 2.2 and 4.1 samples, or 10 mM phosphate buffer for pH 7.2 sample. For pH 1.4 sample, HCl was added to adjust pH.

Similarly, to evaluate the effect of excipients on the peptide stability, peptide solution (1 mg/mL) in 10 mM sodium phosphate buffer (pH 7.4) was dialyzed against the same buffer at 4° C. for overnight and diluted to 150 μg/mL with additive solution in the same buffer.

All samples were incubated at 37° C. for 6-7 days. The peptides remained soluble were detected by Coomassie assay (see below). The integrity of the peptide was examined by fluorescent microscopy or SDS-PAGE (see below).

Solid state stability studies The peptide solution in 10 mM sodium phosphate buffer (pH 7.4), 1 mg/mL, was dialyzed against the same buffer at 4° C. for overnight and diluted to 150 μg/mL with water. Various additives were added at a weight ratio of excipient:peptide=5:1 and the mixture was lyophilized for 2 days. The lyophilized excipient/peptide samples were incubated at 37° C. in a desiccator containing a saturated K₂SO₄ solution, which maintains the relative humidity (R.H.) at 97%. The moisture-wetted samples were removed after incubation for 6-7 days and reconstituted in 10 mM sodium phosphate buffer (pH 7.4) by incubating at 37° C. for 2 hours under mild agitation. The samples were centrifuged at 10,000 r.p.m. for 5 minutes and the supernatant solution was removed to determine remaining soluble peptide and structural integrity.

Protein assays Soluble peptide was quantified by a modified Bradford assay (Coomassie brilliant blue plus protein assay, Pierce Chemical Co.), with the Absorbance read at 595 nm using a Dynex MRX plate reader. The total peptide in microspheres samples was determined by an amino acid assay following complete hydrolysis of the peptide-containing polymer microspheres. Briefly, 7 nmol ornithine was added as internal standard before the samples were completely hydrolyzed in 6 N HCl at 110° C. for 22 hours after sealing under light vacuum. The amino acids were reconstituted in 1 M sodium carbonate buffer (pH 9.5) following removal of hydrochloric acid and derivatized by o-phthaldialdehyde (OPA). The details of the preparation of derivatizing solution and derivatization procedure have been previously described.(21) Leucine from hydrolyzed peptide samples was separated on an ODS column (Nova-Pak® C₁₈, 3.9×150 mm, 4 μm, Waters, Milford, Mass.). A binary gradient mobile phase consisting of 0.05 M sodium acetate buffer, pH 6.8 (eluent A) and 100% methanol (eluent B) was used. The flow rate was 1.5 mL/minute over 22 minutes. The signals were detected by a Waters 474 Scanning Fluorescence Detector (λ_{Ex}/λ_{Em}=340/455 nm).

Evaluation of structural integrity The structural integrity of the peptide was evaluated by sodium dodecylsulfate-polyacrylamide gel electrophoresis (SDS-PAGE) on a High Density PhastGel® performed by a PhastSystem (Amersham Pharmacia Biotech AB, Uppsala, Sweden). Prior to application onto the gel, the peptide samples were mixed with twice concentrated reducing sample buffer containing 1% SDS, 4% 2-mercaptoethanol, 0.02% brilliant blue G and 24% glycerol in 100 mM Tris HCl, pH 6.8. After heating in boiled water for 5 minutes, the mixture was applied to High Density PhastGel® (Amersham Pharmacia Biotech AB) and separated according to Separation File No. 112 provided by the manufacturer. A molecular weight marker kit in ultra-low range (Sigma) was prepared and applied in the same manner. A modified Coomassie brilliant blue staining method using glutadialdehyde as fixing reagent was developed to visualize the low MW peptide bands. For the peptide was extracted from the microspheres (see below), the peptide was reconstituted in PBS. The concentration of the peptide solution was made at 1 mg/mL for all formulations according to the soluble peptide amount known from Coomassie assay (as above).

Fluorescent emission spectra of the peptide were used to monitor the changes of the aromatic amino acid residues. The peptide samples were filtered through a 0.45 μm Milipore filter before the emission spectra (290-500 nm) were obtained. The excitation wavelength was set at 274 nm, increment 1 nm and slit width was 5 nm for both excitation and emission light.

Encapsulation of the peptide antigen in PLGA microspheres PLGA microparticles were prepared by a double emulsion (W/O/W)-solvent evaporation methods. PLGA was dissolved in methylene chloride at 700 mg/mL. 1/10 volume of peptide solution was added and the mixture was homogenized at 10,000 rpm for 1 minute over an ice bath. 5% PVA was added and further homogenized at 10,000 rpm for 1 minute to from w/o/w emulsions. The particles were hardened in PBS solution containing 0.5% PVA for 2 hours under stirring. All microparticles were collected by centrifugation and washed with water and freeze-dried. For freeze-drying, samples were flash frozen in liquid nitrogen and placed on Labcono freeze dry system (Kansas City, Mo.) at 133×10⁻³ mBar or less at a condenser temperature of −46° C. for at least 24 hours.

Determination of the peptide loading. A known amount of antigen-encapsulated microspheres was suspended in acetone and the dissolved polymer was removed by centrifugation. The precipitated peptide pellet was washed twice and acetone was allowed to evaporate before the peptide pellet was reconstituted in PBS solution. The peptide was allowed to dissolve by incubating at 37° C. for 1 hour before the soluble peptide content was quantified by Coomassie assay.

Evaluation of release and stability of the peptide encapsulated in microspheres Around 3-4 mg mps was incubated in 1 mL PBS containing 0.02% Tween 80 (PBST) at 37° C. (n=3). The release media were replaced everyday to avoid interference of aggregate and the microsphere samples were removed at different time intervals. The peptide remained soluble in the microspheres was determined by Coomassie assay after extracted from the polymer as did in Determination of Loading Section. The total peptide remained was evaluated by the amino assay as described in Protein Assay Section. The structural integrity of the peptide was evaluated by SDS-PAGE.

Results and Discussion

Stability of CTP37-TT2 in solution at physiological and simulated polymer microclimate pH When administered as a vaccine, CTP37-TT2 peptide released from the adjuvant formulation inevitably encounters body fluids of neutral pH. In addition, as the commonly used pH condition for in vitro studies of proteins, the peptide stability at neutral pH is important for the guidance of sample handling and set-up of in vitro experiments. Furthermore, the peptide was encapsulated in PLGA microspheres in order to achieve controlled release of the antigen and to induce strong antibody response. Acidic microclimate pH has been known to occur in PLGA microspheres and exhibited detrimental effect on protein stability. Therefore, the pH dependence of this synthetic peptide antigen was evaluated in the first place from acidic to neutral pH. Formation of insoluble aggregation and destruction of the structural integrity of the peptide were monitored by protein assay and SDS-PAGE, respectively. A significant amount (˜70%) of aggregates formed in the peptide solution (1 mg/mL) incubated at slightly acidic (pH 4.1) to neutral pH. For peptide incubated at pH 4.1, the peptide aggregates may be dissolved by the denaturing and reducing reagent (8 M urea and 10 mM DTT). However, for those incubated at neutral pH, the aggregates did not dissolute by the denaturing and reducing solution within hours. It is possible that aggregates of the peptide formed at pH 7.2 have tight packing and dissolution of the aggregates need longer time. For peptide incubated at acidic pH (1.4 & 2.2), all peptides remained soluble following 7-day incubation as detected by Comassie assay.

The structural integrity of the peptide incubated in solution of acidic pH was partially lost. SDS-PAGE studies showed the band corresponding to the peptide monomer either weakened or disappeared at pH 1 and 2. Broadening of bands mostly towards low MW direction was observed. The concentration of the peptide prior to application was made to 1 mg/mL according to the total peptide amount. Worthy of mention, the peptide incubated at pH 7.2 displayed the lowest intensity, which is consistent with aggregation of the peptide and its retarded recovery under denaturing and reducing condition. The aggregation kinetics of the peptide in solution of pH 7.2 during incubation at 37° C. at a lower concentration (0.15 mg/mL) was also monitored for up to 7 days. The onset of aggregation started within 1-3 days. After 5 days, ˜60% of the peptide aggregated. In summary, the peptide is not stable in dilute solution state (0.15-1 mg/mL) regardless of the pH. In solution of acidic pH, the peptide tends to hydrolyze and is unable to maintain the structural integrity. Whereas, at pH 4-7, aggregation of the peptide is a serious problem, with more than 70% aggregated following 1-week incubation at 37° C.

Stabilization of the peptide in solution by excipients A variety of excipients, including sugars, polylols and amino acids, have been known to stabilize proteins and peptide and the addition of stabililizing excipients in protein formulation to maintain its biological function is a common practice. In this study, the stabilization of the hCG peptide is desirable both in solution and in PLGA microspheres. Hence, stabilization of the hCG peptide in solution was attempted with the aid of commonly used protein stabilizers. Depending on the specific protein/peptide, certain excipients may stabilize proteins/peptides to a greater extent than others. The stabilizing effects of sugars, polylols, surfactants, amino acids and some other additives was tested by co-incubating with the peptide in solution (pH 7.4). As shown in Table 4, sugars, both sucrose and sorbitol, only stabilized the peptide at high weight ratio (excipients/peptide=100/1) and greatly enhance the percentage of peptide remained soluble (˜65%). Poloxamer surfactants seemed have no effect on the aggregation of the peptide. The most stabilizing effect was observed from addition of Arginine (100/1) and Glycine (100/1), with around 90% of the peptides remained soluble. Val, His and Cys also helped protect the peptide and the peptide remains soluble was ˜65%. However, the addition of Cys acidify the solution pH to <2, where peptide was found to hydrolyze. His showed promising stabilizing effect at a low weight ratio of 10/1, though, the fluorescent spectra displayed complete loss of fluoresce of the peptide, which suggesting a substantial changes in the peptide's primary structure. No significant changes of fluorescent emission spectra of the peptide co-incubated with Arg and Gly was observed. The mechanism of the stabilizing effect of sodium azide remains unknown. The combination of sugar, amino acids and sodium azide completely stabilized the peptide for as long as 1 week at low concentration (150 μg/mL).

TABLE 4
Effect of excipients on the stability of hCG peptide (150 mg/mL)
in solution at neutral pH following incubation at 37° C.

| | | | Soluble peptide, % |
|---|---|---|---|
| Additives | Incubation | pH of | Mean ± SD |
| (Excipient/peptide, w/w) | Time (Days) | solution | n = 3 |
| No excipients | 7 | 7.4 | 9.0 ± 3.7 |
| Sugars and polyols | | | |
| Sucrose (10/1) | 7 | 7.4 | 7.3 ± 3.4 |
| Sucrose (100/1) | 7 | 7.4 | 64.4 ± 5.7 |
| Sorbitol (10/1) | 7 | 7.4 | 10.7 ± 0.9 |
| Sorbitol (100/1) | 7 | 7.3 | 65.1 ± 6.6 |
| Surfactants | | | |
| Poloxamer F38 (10/1) | 7 | 7.4 | 10.0 ± 5.7 |
| Poloxamer F38 (100/1) | 6 | 7.3 | 15.8 ± 8.0 |
| Poloxamer L31 (10/1) | 7 | 7.3 | 10.2 ± 2.2 |
| Amino Acids | | | |
| Valine (10/1) | 7 | 7.3 | 21.4 ± 1.5 |
| Valine (100/1) | 6 | 7.3 | 65.4 ± 3.1 |
| Leucine (10/1) | 7 | 7.3 | 20.7 ± 3.2 |
| Leucine (100/1) | 6 | 7.2 | 36.7 ± 3.0 |
| Arginine (100/1) | 6 | 7.0 | 92.0 ± 5.5 |
| Glycine (100/1) | 6 | 7.2 | 87.6 ± 5.5 |
| Histadine (10/1) | 7 | 7.4 | 64.8 ± 3.7 |
| Glutamic acid (10/1) | 7 | 4.7 | 8.9 ± 5.3 |
| Cysteine (100/1) | 6 | 1.8 | 60.5 ± 7.2 |
| Others | | | |
| PEG, M_{w} 4,600 (10/1) | 7 | 7.4 | 12.9 ± 2.8 |
| Na azide, 0.02% (w/v) | 6 | 7.3 | 87.6 ± 4.4 |
| Mg lactide (10/1) | 7 | 7.0 | 12.3 ± 6.3 |

Solid state stability of the peptide. The physical state of peptide encapsulated in PLGA microspheres was thought to be in between of solution and solid state, depending on the solubility and loading of the peptide. Hence, the stability of the peptide in the solid state was also evaluated to provide simulation of peptide stability. The solid state stability here refers to the stability of peptide powder lyophilized from solution at a certain pH after incubation in elevated temperature (37° C.) and moisture (R.H. 97%, from saturated K₂SO₄ solution). The peptide powder take up moisture from the environment and may exist as a saturated solution with co-existing solid remaining undissolved. As shown in Table 5, the peptide was more stable in the solid state. Following 6-day incubation at 37° C. and 97% R.H., only ˜10% aggregation of the peptide was detected when lyophilized from pH 7.4, which may be explained by the reduced mobility of peptide chain and bound water. Amino acids which exhibited stabilizing effect on the peptide in dilute solution, such as Val, Leu, Arg, and Gly, showed similar protection toward peptide in the solid state. The peptide was recovered almost completely in the presence of stabilizing additives. Cysteine, on the other hand, destabilize the peptide, with only ˜20% of peptide remained soluble as detected by Coomassie assay.

TABLE 5
Effect of excipients on the stability of hCG peptide in solid state,
following 6-day incubatoin at 37° C., 97% relative humidity (RH).

| Additives | Soluble Peptide, % |
|---|---|
| (excipient:peptide 5:1. w/w) | M ± SD, n = 3 |
| No excipients | 89.5 ± 6.6 |
| Amino acids | |
| Tyrosine | 92.6 ± 8.3 |
| Valine | 100.6 ± 4.2 |
| Leucine | 102.0 ± 1.1 |
| Arginine | 105.3 ± 2.2 |
| Glycine | 94.5 ± 7.5 |
| Glutamic Acid | 105.6 ± 3.9 |
| Cysteine | 18.0 ± 0.2 |
| Surfactants | |
| Poloxamer F38 | 105.5 ± 3.1 |

^{a}Surface-conjugated formulation (SCF): PLGA microsphere formulation with the peptide conjugated on the surface.
^{b}Encapsulated formulation (EnF): PLGA microsphere formulation with the peptide encapsulated.
^{c}The soluble peptide in PBS solution was administered as a negative control.
^{d}The peptide was incorporated in an water-in-oil (PBS-in-squalene:mannide monooleate (4:1) (40:60)) emulsion.
^{e}nor-MDP was encapsulated in 0.624 mg PLGA microspheres.
^{f}nor-MDP solution was used.

The structural integrity of the hCG peptide lyophilized from pH 2 was determined by SDS-PAGE. Reduced hydrolysis of the peptide in the solid state occurred when exposed to elevated temperature and moisture, compared with peptide in dilute solution at similar pH. The peptide sample following incubation at 37° C. for 14 days exhibited decreased intensity where corresponds to the peptide monomer. Though the band was still visible, as opposed to the peptide sample incubated in solution. Interestingly, aggregation of the peptide seemed precedate the fragmentation of the peptide at acidic pH.

Stability of the peptide in PLGA microspheres In our previous study, the CTP37-TT2 antigen was encapsulated in PLGA microspheres in order to provide sustained antigen release and to induce effective antibody responses. The stability is a main issue for peptide encapsulated in PLGA microspheres and is essential for controlled release of the antigen. Herein, the stability of this synthetic hCG immunogen in the polymer was examined. The homogenization condition during microencapsulation was suited to produce particles with size within the range of 1-15 μm. As shown in Table 6, all four microparticle formulations exhibited spherical microspheres with smooth surface. The addition of the excipients did not influence the particles size and morphology of the microspheres. The peptide loading was around 0.5-0.8% and the loading efficiency was around 55-70% for the peptide. The co-encapsulation of excipients, especially Arginine which is added in the peptide solution, decrease the encapsulation efficiency.

TABLE 6
Characteristics of the PLGA microspheres.

| | | | | Burst |
|---|---|---|---|---|
| | | Loading, % | Encap- | Release |
| For- | | (mean ± SD, | sulation | (within 1 |
| mulation | Excipients | n = 3) | Efficiency | day) |
| A | No excipients | 0.76 ± 0.09 | 70.4% | 4.1% |
| B | 3% MgCO₃ | 0.69 ± 0.03 | 63.9% | — |
| C | 3% Arginine | 0.52 ± 0.03 | 48.1% | 15.5% |
| D | 3% MgCO₃ + 3% Arg | 0.61 ± 0.01 | 56.5% | 33.5% |

^{a}Group I-VIII as described in Table 2.
^{b}Blank microspheres. Neither antigen nor MgCO3 was encapsulated;
^{c}Blank microspheres. 3% MgCO₃ was added during microsphere preparation. No antigen was present.

Besides particle size and loading, the burst release of the peptide was significantly influenced by the addition of the excipients. MgCO₃ alone seems did not increase the burst release of the peptide within 1 day, whereas, Arginine and combination of arginine and MgCO₃ significantly enhance the burst release, probably due to the increased osmotic pressure by the excipients. Continuous release of the peptide from the microspheres was observed over a month in Formulation A (no excipients), B (with 3% MgCO₃) & D (with 3% MgCO₃ and 3% Arginine).

The aggregation of the peptide in the microspheres was evaluated from the comparison of the total peptide retained and peptide remained soluble in the microspheres after incubation in the release media for certain period of time. It was found that in formulations without any excipients, around 70% of the peptide remained soluble after 33 days. A slight decrease in the aggregation rate of the peptide (20% vs. 30% after 33 days) in the polymer was observed in the formulation with 3% MgCO₃. Surprisingly, the presence of arginine causes the peptide aggregation as seen in Formulations C and D.

The peptide stability was further analyzed regarding hydrolysis after extraction from the polymer by SDS-PAGE. After 33-day incubation in the release media, most peptide remained unhydrolyzed in formulation A. A broadening in the band suggests the potential fragmentation may occur later. However, in the formulations with excipients, especially in formulations with Arginine (C & D), decrease in the band intensity along with broadening of the band were more obvious.

### CONCLUSIONS OF EXAMPLE 2

Stability of the encapsulated peptide more closely mimicked stability in the wetted solid-sate as compared to the dilute solution. These stability results may serve as stability guidelines for handling of the peptide in solution and for its potential use as a slow-release birth control vaccine.

### EXAMPLE 3

### Immunogenicity testing of a microsphere formulation containing a synthetic and an organic salt.

Materials Microspheres containing a synthetic peptide representing the carboxyl terminal 35 amino acid residues of the beta subunit of hCG [on the C-terminus]co-synthesized with an amino acid sequence of a T-cell lymphocyte epitope of tetanus toxoid or measles protein (on the N-terminus) with and without a quantity of MgCO₃ incorporated into the lactide/glycolide polymer were tested for their ability to elicit sustained high levels of antibodies reactive with the intact hCG molecule. Also tested was a conjugate of a peptide covalently linked to microspheres encapsulated with polylysine. The preparations tested are provided in Table 7.

TABLE 7
Composition of Preparations Tested

| | | Antigen | Adjuvant | MgCO₃ | Physical Form |
|---|---|---|---|---|---|
| Group | Antigen | Dose (mg) | Dose (mg) | (mg) | of Microspheres |
| 1 | TT-hCG | 0.200 | 0 | 0 | Conjugate |
| 2 | TT-hCG Peptide | 1.0 | 0 | 4.29 | Encapsulated |
| 3 | TT-hCG Peptide | 1.0 | 0 | 4.29 | Combination of |
| | | | | | conjugate and |
| | | | | | encapsulated peptide |
| 4 | TT-hCG Peptide | 0.200 | 0.25 nor MDP | 0 | Conjugate |
| 5 | TT-hCG Peptide | 1.0 | 0.25 nor MDP | 4.29 | Enclapsulated |
| 6 | TT-hCG Peptide | 1.0 | 0.25 nor MDP | 4.29 | Combination of |
| | | | | | conjugate and |
| | | | | | encapsulated peptide |
| 7 | TT-hCG Peptide | 1.0^{a} | 0.25 each | 0 | A water-in-oil emulsion - |
| | | | injection | | no microspheres |
| 8 | TT-hCG Peptide | 1.0 | 0.25 nor MDP | 0 | Peptide dissolved in |
| | | | | | PBS - no microspheres |
| 9 | MVF-hCG | 1.0 | 0 | 0.48 | Encapsulated |
| | Peptide | | | | |
| 10 | MVF-hCG | 1.0 | 0 | 0 | Encapsulated |

^{a}Antigen dose was 1.0 mg three times (at 0, 3, and 6 weeks)

Methods Animals—testing for antibody production was done using adult, specific pathogen-free New Zealand White rabbits. They were housed in a temperature-controlled room and water and food ad libitum.

Immunizations Dry microspheres were suspended in phosphate-buffered saline (0.05 M sodium phosphate and 0.15 M sodium chloride, pH 7.2) (PBS) in a concentration that yielded 1.0 mg peptide per mL of PBS. One mL of the suspension was injected subcutaneously into the thigh muscle of the rabbit using a sterile technique. Only one immunization per animal was performed, and the day of the immunization was designated as time 0. Control animals groups were immunized as described in Results.

Serum Collections Beginning on day 14 from immunization, blood samples were collected weekly by venipuncture of an ear vein. The blood was allowed to clot at room temperature for one hour, and the serum separated from the cells by centrifugation. Serum was aspirated and stored in glass vials at −20° C. until tested for antibody content.

Antibody Testing The antibody concentration in collected sera was tested by a competitive radioimmunoassay employing I¹²⁵-labeled highly purified hCG using the method described by Powell et al.[20] Briefly, the method consists of reacting a quantity of diluted serum (in PBS) with 20-40 ng of radio-iodinated hCG alone, and with a range of amounts of unlabelled hCG. The mixture was incubated at 4° for 96 hours, brought to room temperature, and the hCG-antibody complexes precipitated by the addition of an amount of polyethylene glycol (PEG). For each sample assayed, a downward curve of binding displacement of labeled hCG is created and the resulting negative regression curve subjected to Scathard analysis.[21] The quantity of antibody is estimated from this analysis and expressed as nanomoles binding/milliliter of serum (nM/mL).

RESULTS Antibody Levels Table 7 describes the ten groups of rabbits immunized with various lots of microspheres containing either 1.0 mg of peptide or a peptide-microsphere conjugate or combinations of the two together with or without the co-incorporation of a quantity of magnesium carbonate. Positive and negative control groups are listed as Groups 9 and 10. Initially, Groups 1-6 were studied. Groups 1 and 4 rabbits were immunized with a conjugate of a peptide covalently linked to pre-formed microspheres containing polylysine by a method patented by Schwendeman. Both groups received 200 micrograms of TT-hCG peptide and Group 4 also received 25 micrograms of a synthetic adjuvant compound (nor MDP) encapsulated in lactide/glycolide microspheres. The two kinds of microspheres were mixed and administered at the same time. Both groups elicit significant levels of antibodies for several weeks, but these levels were not considered superior to those found using conventional methods of immunization. There was no enhancement in levels in Group 4 over those found in Group 1 suggesting the synthetic adjuvant did not augment the immune response to the peptide All antibody levels were determined as the mean of 5 animals unless otherwise indicated.

Groups 2 and 5 received microspheres containing 1.0 mg of the TT-hCG peptide incorporated into the polymer to which was added 3% MgCO₃. The peptide load (concentration) in these microspheres was such that quantity of particles containing 1.0 mg of peptide contained 4.29 mg of MgCO₃. Group 5 rabbits also received the synthetic adjuvant in separate microspheres.

The results from these latter immunizations were determined, and showed that both groups of animals produced very high levels of antibodies relative to conventional methods and the levels were maintained for several months. The levels were still elevated at 24 weeks when the study was terminated. Group 2 levels (without adjuvant) were slightly higher than those found in group 5 (adjuvant added), but the difference was not considered to be significant. Again, these results suggested that the adjuvant had little or no effect on the production of high antibody levels.

Rabbits in Groups 3 and 6 were combinations of the microspheres given in Group 1 plus Group 2 and Group 4 plus Group 5, respectively. The antibody levels attained in animals in these two groups exceeded the already excellent levels attained by Groups 2 and 5 rabbits. These data suggest that the addition of conjugate microspheres to the microspheres containing peptide plus magnesium carbonate gave an augmented or additive response over the use of the latter particles alone. Again, the group (Group 6) containing the synthetic adjuvant did not produce antibody levels significantly greater than the group without it (Group 3).

The significance of these findings is revealed when one compares the level and duration of antibodies elicited by either conventional immunization methods using three injections (Group 7), or administering the TT-hCG peptide dissolved in PBS only (Group 8). The PBS group produced very low levels, although they were sustained for a rather long time. The animals immunized by a conventional method (peptide dissolved in PBS and emulsified with squaline/mannide monooleate [4:1] in a ratio of PBS:S/MM of 1:1 and 1.0 mg of peptide in 1.0 mL of emulsion injected three times at 0, 3, and 6 weeks), elicited moderate levels of antibody, but not nearly as high and as sustained as the single-injected rabbits receiving microspheres containing the same peptide plus magnesium carbonate injected in PBS as a vehicle. Thus, these results clearly indicate that the peptide incorporated into microspheres containing magnesium carbonate can elicit antibody levels superior to conventional methods and these can be sustained for a protracted period after a single administration.

The amount of magnesium carbonate injected with each 1.0 mg of peptide in these experiments (Groups 2, 3, 5, and 6) was 4.29 mg. As levels of antibodies attained in these groups were higher than those theoretically needed for an hCG therapeutic vaccine, an experiment was conducted using a lower amount of the salt per each 1.0 mg of peptide. For this study, only the IVF-hCG peptide was available, but it was known to be equally immunogenic as the TT-hCG peptide using conventional methods of immunization. In this study, lactide/glycolide microspheres containing 1.4 mg of peptide and a magnesium carbonate level of 0.48 mg, nearly 10 times less MgCO₃ than the earlier studies was used. Only three rabbits were used in this experiment. Antibody levels were not as high as those seen in earlier studies using the TT-hCG peptide and higher levels of MgCO₃, but were much higher than the control (PBS injected) group and nearly as high as those found in animals immunized by conventional methods. These findings suggest, although two different peptides were used, that the level of MgCO₃ is important for the production of high antibody levels. This suggestion was confirmed when 1.0 mg of the same IVF peptide was incorporated into microspheres without MgCO₃. The levels of antibodies attained by these rabbits in this group (Group 10) were much lower than those receiving 1.0 mg of the peptide together with 0.48 mg of the salt (Group 9).

Taken together, these data provide evidence that the incorporation of MgCO₃ into PLGA microspheres containing peptide antigens, prepared by the standard methods, enhances the production of antibodies to the peptide following a single immunization with the particles suspended in PBS.

### REFERENCES

1. Stevens, V. C., et al., Preparation and formulation of a human chorionic gonadotropin antifertility vaccine: selection of a peptide immunogen. Am. J. Reprod. Immunol., 1981. 1: p. 307-314.

2. Stevens, V. C., Action of antisera to hCG-beta. in Fifth International Congress of Encocrinology.

3. Zauber, W., et al., Defined Synthetic vaccines. Biol Chem, 2001. 382: p. 581-595.

4. Stevens, V. C., Progress in the development of human chorionic gonadotropin antifertility vaccines. American J Reprod Immunol, 1996. 35: p. 148-155.

5. P, p.-b., universally immunogenic T epitopes promiscous binding to human MHC II and recognition by T cells. European J immunology. 19: p. 2237.

6. Kaumaya, Peptide vaccines incorporating a promiscous t cell epitope bypass certain haplotype restricted immune responses and provide broad spectrum immunogenicity. J molecular recog, 1993.

7. Johansen, P., et al., Revisiting PLA/PLGA microspheres: an analysis of their potential in parenteral vaccination. European J Pharm Biopharm, 2000. 50: p. 129-146.

8. Bloom, B. R., Vaccine for the Third World. Nature, 1989. 342: p. 115-120.

9. Cleland, J. L., Design and production of single-immunization vaccines using polylactide polyglycolide microsphere systems, in Vaccine design: the subunit and adjuvant approach, M. F. Powell and M. J. Newman, Editors. 1995, Plenum Press: New York. p. 439-462.

10. Eldridge, J. H., Biodegradable and biocompatible poly(DL-lactide-co-glycolide) microspheres as an adjuvant for staphylococal enterotoxin B toxoid which enhances the level of toxin-neutralizing antibodies. Intect. Immun., 1991. 59: p. 2978-86.

11. Men, Y., et al., Induction of cytotoxic T lymphocyte response by immunization with a Lalaria specific CTP peptide entrapped in biodegradable polymer microspheres. Vaccine, 1997. 15: p. 1405-1412.

12. Men, Y., et al., MHC class I and class II restricted processing and presentation of microencapsulated antigens. Vaccine, 1999. 17: p. 1047-1056.

13. Jondal, M., R. Schirmbeck, and J. Reimann, MHC class I restricted CTL responses to exogenous antigens. Immunity, 1996. 5: p. 295-302.

14. Sedlik, C., Antigen linked to synthetic microspehres induce immune reponses in primates in the absence of adjuvants. Immunobio, 1996.

15. Gengous, C. and C. Leclerc, In vivo induction of CD4⁺ T cell responses by antigen covalently linked to synthetic microspehres does not require adjuvants. 1995.

16. J. E., P., et al., Characteristics of Antibodies Raised to Carboxy-Terminal Peptides of hCG Beta Subunit. J. Reprod Immunol, 1980. 2: p. 1-13.

17. Shenderova, A., T. G. Burke, and S. P. Schwendeman, The acidic microclimate in poly(lactide-co-glycolide) microspheres stabilizes camptothecins. Pharm. Res., 1999. 16(2): p. 241-8.

18. Zhu, G., Stabilizaion and Controlled Release of Proteins Encapsulated in Poly(lactide-co-glycolide) Delivery Systems, in Pharmaceutics. 1999, Ohio State University: Columbus. p. 9.

19. Lee, A. C. J., et al., A method for preparing B-hCG COOH peptide-carrier conjugates of predictable composition. Mol. Immunol., 1980.17: p. 749-756.

20. Powell J. E., A. C. Lee, G. W. Tregear, H. D. Niall, and V. C. Stevens, Characteristics of antibodies raised to carboxy-terminated peptides of hCG Beta Subunit. Journal of Repro Immuno, 1980, 2: pp. 1-16.

21. Scatchard, G., The attractions of proteins for small molecules and ions. Ann. N. Y. Acad. Sci. 51: pp. 660-672.

The invention claimed is:

1. A method of enhancing an immunogenic response in a mammalian subject, the method comprising administering a biodegradable polymeric delivery system comprising a biologically effective amount of one or more antigens and one or more basic additives to the mammalian subject.

2. The method of claim 1 wherein the antigen is selected from the group consisting of nucleic acids, proteins, polypeptides, peptides, polysaccharides, hapten conjugates, and combinations thereof.

3. The method of claim 2 wherein the antigen is a peptide.

4. The method of claim 1 wherein the basic additive is characterized by having a pH of a saturated solution at 37° C. in the range from about 6.8 to about 12.5 and a solubility in water at 37° C. from 1.2×10⁻² to about 3×10⁻¹¹.

5. The method of claim 1 wherein the basic additive is selected from the group consisting of magnesium carbonate, magnesium hydroxide, magnesium oxide, magnesium trisilicate, zinc carbonate, zinc hydroxide, zinc phosphate, aluminum hydroxide, basic aluminum carbonate, dihydroxyaluminum sodium carbonate, dihydroxyaluminum aminoacetate, ammonium phosphate, calcium phosphate, calcium hydroxide, magaldrate, calcium sulfate and combinations thereof.

6. The method of claim 1 wherein the mammalian subject is a human.

7. A method of enhancing an immunogenic response to human chorionic gonadatropin (hCG) in a subject, the method comprising administering a biodegradable polymeric delivery system comprising a biologically effective amount of an hCG antigen and a basic additive to the subject.

8. The method of claim 7 wherein the hCG antigen is a carboxyl terminal peptide (CTP) of hCG.

9. The method of claim 7 wherein polymeric delivery system comprises from 0.08 to 20% antigen based on the weight of the polymer.

10. The method of claim 7 wherein the antigen is conjugated to the polymeric delivery system and encapsulated in the polymeric delivery system.

11. The method of claim 7 wherein the antigen is conjugated to the polymeric delivery system.

12. The method of claim 7 wherein the antigen is encapsulated in the polymeric delivery system.

13. The method of claim 7 wherein the basic additive is characterized by having a pH of a saturated solution at 37° C. in the range from about 6.8 to about 12.5 and a solubility in water at 37° C. from 1.2×10⁻² to about 3×10⁻¹¹.

14. The method of claim 7 wherein the basic additive is selected from the group consisting of magnesium carbonate, magnesium hydroxide, magnesium oxide, magnesium trisilicate, zinc carbonate; zinc hydroxide, zinc phosphate, aluminum hydroxide, basic aluminum carbonate, dihydroxyaluminum sodium carbonate, dihydroxyaluminum aminoacetate, ammonium phosphate, calcium phosphate, calcium hydroxide, magaldrate, calcium sulfate and combinations thereof.

15. The method of claim 14 wherein the basic additive is MgCO₃.

16. The method of claim 7 wherein the ratio of basic additive to antigen is from 0.5:1 to 30:1 (w/w).

17. The method of claim 16 wherein the ratio of basic additive to antigen is about 4:1 (w/w).

18. The method of claim 7 wherein the ratio of basic additive to biodegradable polymer is from 0.5 to 20% (w/w).

19. The method of claim 18 wherein the ratio of basic additive to biodegradable polymer is from 1 to 7% (w/w).

20. The method of claim 7 wherein basic additive is added at a level of 3% or less based on the weight of the polymer.

21. The method of claim 7 wherein the biodegradable polymeric delivery system is a poly(lactide-co-glycolide) (PLGA) delivery system.

22. The method of claim 21 wherein the PLGA is poly(D-L-lactide-co-glycolide).

23. The method of claim 21 wherein the ratio of lactide/lactic acid to the ratio of glycolide/glycolic acid is in the range from 100:0 to 0:100.

24. The method of claim 23 wherein the ratio of lactide/lactic acid to the ratio of glycolide/glycolic acid is in the range from 100:0 to 50:50.

25. The method of claim 7 wherein the PLGA polymeric delivery system further comprises an adjuvant.

26. The method of claim 7 wherein the PLGA delivery system further comprises an excipient.

27. An immunogenic composition for eliciting an immune response against an antigen comprising:
a) a biodegradable polymeric delivery system;
b) a biologically effective amount of an antigen; and
a) a basic additive.

28. An immunogenic composition for eliciting an immune response against human chorionic gonadatropin (hCG) comprising:
a) a poly(lactide-co-glycolide) polymeric delivery system; wherein the ratio of lactide/lactic acid to the ratio of glycolide/glycolic acid is in the range from 100:0 to 50:50;
b) 0.08 to 20% (w/w) of an hCG antigen, based on the weight of the polymer, wherein the hCG antigen is a carboxyl terminal peptide (CTP) of the beta subunit of hCG; and
c) 0.5 to 20% (w/w) of a basic additive, based on the weight of the polymer, wherein the basic additive is selected from the group consisting of magnesium carbonate, magnesium hydroxide, magnesium oxide, magnesium trisilicate, zinc carbonate, zinc hydroxide, zinc phosphate, aluminum hydroxide, basic aluminum carbonate, dihydroxyaluminum sodium carbonate, dihydroxyaluminum aminoacetate, ammonium phosphate, calcium phosphate, calcium hydroxide, magaldrate, calcium sulfate and combinations thereof.

29. The immunogenic composition of claim 28 wherein the ratio of basic additive to antigen is about 4:1.
